(19) United States
(12) Patent Application Publication
(10) Pub. No.: US 2021/0018517 A1
(43) Pub. Date: Jan. 21, 2021

(54) METHOD FOR THE DIAGNOSIS OF C3NEF-MEDIATED MEMBRANOPROLIFERATIVE GLOMERULONEPHRITIS

(71) Applicant: PARCO SCIENTIFICO TECNOLOGICO KILOMETRO ROSSO SPA, BERGAMO (IT)

(72) Inventors: ROBERTA DONADELLI, BERGAMO (IT); MARINA NORIS, BERGAMO (IT); ARIELA BENIGNI, BERGAMO (IT); GIUSEPPE REMUZZI, BERGAMO (IT)

(21) Appl. No.: 17/043,805

(22) PCT Filed: Mar. 29, 2019
(86) PCT No.: PCT/IB2019/052603

§ 371 Date: Sep. 30, 2020

(30) Foreign Application Priority Data
Mar. 30, 2018 (IT) 102018000004141

(51) Int. Cl. G01N 33/68; G01N 33/564

(52) U.S. Cl. CPC G01N 33/6893; G01N 2800/347; G01N 33/564

(57) ABSTRACT

A method for the diagnosis of C3NeF-mediated membranoproliferative glomerulonephritis (MPGN) in a sample may include the step of immobilization of C3b in at least two wells of a plate. The method may further include formation in the wells of the C3bBb complex, where the formation of the complex is mediated by the addition of IgGs purified from the sample and/or control in the presence of Factor B (FB), Factor D (FD), bovine serum albumin (BSA), or magnesium ions. The method may also include detachment of the formed complexes and an electrophoretic run under denaturing conditions of the complexes and transfer on a membrane. Further, the method may include detection of the protein of interest, Bb. The ratio of the intensities measured for the Bb band in the samples collected by each of the wells is indicative of C3NeF activity in the sample.

The present invention relates to a method for the diagnosis of C3NeF-mediated membranoproliferative glomerulonephritis in a subject. The method comprises:

a) immobilization of C3b in at least two wells of a plate;

b) formation in said wells of the C3bBb complex representing the C3 convertase of the alternative pathway, where said formation of the complex is mediated by the addition of IgGs purified from a biological sample isolated from the patient and/or healthy subject, in the presence of Factor B (FB), Factor D (FD), Bovine Serum Albumin (BSA), magnesium ions;

c) detachment of the formed complexes;

d) electrophoretic run under denaturing conditions of said complexes and transfer on a membrane;

e) detection of the protein of interest, Bb, which is one of the two components, C3b and Bb, of C3 convertase; where the ratio of the intensities measured for the Bb band in the samples collected by each of said wells is indicative of C3NeF activity in said subject.

### BACKGROUND ART

Membranoproliferative glomerulonephritis (MPGN) is a chronic progressive nephropathy framed in the context of a nephrotic syndrome and characterized by lesions of the glomerular filtration membrane and intense proliferation of glomerular cells.

Underlying the nephropathy there is a hyperactivation of the alternative pathway of the complement, caused either by genetic alterations in the complement genes (genetic forms) or by acquired alterations (acquired or autoimmune forms) due to the presence of auto-antibodies, called C3 nephritic factors (C3NeFs). About 50-80% of patients with MPGN have C3NeFs.

In MPGN, low plasma C3 levels due to the consumption of this factor are observed, caused by the activation of the alternative complement pathway.

The activation of the alternative pathway leads to the formation of C3 convertase, responsible for the cleavage of C3 to C3b. Under normal conditions, the cleavage of circulating C3 occurs at a very slow pace, with the formation of very small amounts of C3b. This, if it remains in circulation, is rapidly inactivated to iC3b by serine protease Factor I; if instead it binds to the surface of foreign cells, for example bacterial cells, it can be associated with a plasma protein called Factor B (FB). C3b binds to FB forming the pro-enzyme C3 proconvertase, C3bB. In this complex, FB becomes susceptible to the cleavage of the Ba fragment by a circulating protease called Factor D. The residual fragment, Bb, remains bound to C3b constituting the C3bBb complex representing the active C3 convertase, C3bBb, of the alternative pathway. The C3 convertase represents the key enzyme of the alternative complement pathway as it is able to cleave large amounts of C3 in C3b and C3a, leading to a rapid amplification of the activation process of the alternative complement pathway, (amplification loop of the formation of C3 convertase). To avoid an excessive activation of the complement alternative pathway, this cascade, in physiological conditions, is mantained strictly under control by a series of regulators both circulating and bound to the membrane of the body's own cells. The Factor H (FH), a plasma protein, has a fundamental role in the regulation of the alternative complement pathway both in the circulation and on the cell surface. FH determines the dissociation of C3 convertase (FH-mediated decay) and acts as a cofactor of Factor I in inactivating C3b (iC3b).

The only positive regulator of the alternative complement pathway is represented by properdin, a plasma protein which, by binding to the C3bBb complex, stabilizes it increasing its half-life.

C3NeF is an IgG auto-antibody directed against the antigenic determinants expressed on C3bBb. C3NeF binds C3bBb and this bond results in a stabilization of the C3bBb complex. C3NeF bound to C3 convertase prevents both spontaneous decay and FH-accelerated decay. The presence of C3NeF and its ability to stabilize the complex is a parameter for the diagnosis of MPGN. From a technical point of view, the analysis of C3NeF still presents strong problems. The method traditionally used to evaluate the presence and activity of C3NeF is based on the hemolysis of sheep red blood cells in the presence of complement factors and IgGs isolated from patients (Fremeaux-Bacchi V et al. Nephrology Dialysis Transplantation, 9 (1994) 1747-1750). It is a functional test evaluating the final effect of the activation of the complement system and does not allow to study the activation mechanisms.

Jin Seino et al., in Journal of Immunological Methods 159 (1993) 221-227, disclose a method based on the ELISA assay for C3NeF measurement. The method comprises the immobilization of C3b in ELISA plates, where the C3bBb complex was formed in the presence of nickel ions. The tested sample serum is added to the wells to measure the activity of C3NeF as a function of the amount of IgGs that in the serum sample were bound to the C3bBb complex pre-formed on the plate. The limit of the method is represented by the use of Nickel, ion known in literature to stabilize above all the C3 proconvertase, C3bB, and only to a lesser extent the C3 convertase, C3bBb. With this ELISA assay, it is not possible to distinguish how many IgGs bound to C3bB and how many to C3bBb.

Paixao-Cavalcante et al., in Kidney International 82 (2012) 1084-1092, disclose an ELISA method to evaluate the effect of IgG/C3NeF limitedly to the formation of C3 convertase, i.e. they evaluate how much IgGs cause more or less convertase to form taking as a reference a strongly positive sample for C3NeF. This method does not evaluate the impact of C3NeF on the half-life of the enzyme complex. Said method also does not discriminate between the formation of C3 proconvertase and C3 convertase, where both are detected with a single signal by the same anti-FB antibody.

Bettoni et al., In J Immunol 199 (2017) 1021-1040, disclose a method combining microplate and Western Blot. The method allows to distinguish, due to the electrophoretic separation, the formation of C3 proconvertase, displayed as a band of the whole FB of 93 KDa, and the formation of the C3 convertase, displayed as a band of Bb of 60 KDa. However, the reported method does not describe the evaluation of the C3 convertase decay.

The need is strongly felt for an effective assay for a correct evaluation not only of C3NeF levels but also of C3NeF activity on C3 convertase decay, in an environment as close as possible to the physiological context, so as to allow an accurate diagnosis of the pathology.

### DESCRIPTION OF THE INVENTION

The present invention relates to a combined plate-Western Blot method for evaluating the stabilizing effect of C3NeF, isolated from a patient serum, on C3 convertase of the alternative complement pathway.

### DESCRIPTION OF DRAWINGS

FIG. 1: WB image and relative quantization of the signal, representative of a first embodiment of the method according to the present invention.

FIG. 2: WB image and relative quantization of the signal, representative of a second embodiment of the method according to the present invention.

FIG. 3: WB image and relative quantization of the signal, representative of a third embodiment of the method according to the present invention.

### DETAILED DESCRIPTION OF THE INVENTION

The method according to the present invention comprises the formation, in wells, of C3bBb complexes, the detachment thereof and the evaluation, by Western Blot analysis, of the intensity of the Bb band corresponding to the formation of C3 convertase. Typically, wells of a 96-well plate are used.

In particular, said method comprises the formation of C3bBb complexes in wells, where the formation of said complexes occurs in the presence of Magnesium ions, thus obtaining a defined baseline value. To evaluate the stabilizing effect of C3NeF on C3 convertase, in additional wells, the C3 convertase complexes formed in the presence of IgGs/C3NeFs, are incubated in the absence (spontaneous decay) or in the presence of FH (FH-mediated decay). The stabilizing activity of C3NeF is measured after the detachment of said complexes from the wells and the electrophoretic run thereof, evaluating the ratio of the intensities of Bb before (baseline) and after the decay of said C3bBb complex.

In a first embodiment, the method according to the present invention comprises:

a) immobilization of C3b in the plate wells, preferably a 96 microwell plate;

b) addition in said wells of IgGs purified from a biological sample isolated from a patient and/or healthy subject, in the presence of FB, FD, BSA, and magnesium ions so as to form the C3bBb complex;

c) detachment of the formed complexes;

d) electrophoretic run of said complexes, with dissociation, due to the denaturing conditions (presence of SDS), of the two components of C3 convertase, C3b and Bb, and transfer thereof on a membrane;

e) detection of the protein of interest, Bb, to obtain a defined baseline value

characterized in that to said step b) of addition of purified IgGs there follows, in one or more wells, after a washing step, a further incubation in the presence or in the absence of FH, wherein the ratio of the measured intensity for the Bb band in the samples subjected to said further incubation to the baseline Bb band is indicative of C3NeF activity.

Said method is indicative of a positivity of the tested subject when values of the Bb band intensity are recorded after said further incubation equal to at least 34, or at least 35, preferably at least 37% of the values measured with respect to the baseline of the same sample, and/or values of the Bb band intensity after further incubation in the presence of FH equal to at least 9, or at least 10, preferably at least 12% of the values measured as baseline of the same sample.

Preferably, said immobilization step a) is carried out using purified human C3b (Complement Technology, USA USA) diluted in PBS buffer, with incubation overnight (ON) at 4° C. After washing with PBS, the non-specific sites are blocked, typically incubating in PBS, 1% BSA, 0.1% Tween for 1 h at 37° C. and subsequent washing with washing buffer (8.1 mM Na₂HPO₄, 1.8 mM NaH₂PO₄, 0.1% Tween20, 25 mM NaCl and 10 mM MgCl₂).

Preferably, in said step b) the wells with the immobilized C3b are incubated for 12 minutes at 25° C., with IgGs 100 μg/ml, FB 1000 ng/ml, FD 10 ng/ml in a buffer containing 8.1 mM Na₂HPO₄, 1.8 mM NaH₂PO₄, 0.1% Tween20, 0.5% BSA, 10 mM MgCl₂ and 75 mM NaCl.

Preferably, in said step c) said complexes are detached by incubating in 10 mM EDTA, 1% SDS for 1 h at room temperature (RT).

Preferably, said electrophoretic run of step d) is carried out on 10% polyacrylamide gel with addition of 0.1% SDS (denaturing condition) and under reducing conditions, obtained with 3% beta-mercaptoethanol. At the end of the electrophoretic run, proteins are transferred onto a membrane and said membrane is a PVDF membrane (Biorad, Inc. USA). The proteins are highlighted with the goat anti-human FB primary antibody (Quidel) diluted 1:10000 in tris buffer (TBS) supplemented with 5% non-fat milk and 0.1% Tween20 (blocking buffer), incubating overnight at 4° C., followed by binding with a secondary antibody consisting of anti-goat IgGs conjugated to HRP (Vector Laboratories, 1:10000) for 1 h at room temperature. The signal is read with the ECL system (Amersham).

The quantification of the band intensity (densitometric analysis) of 60 kDa, corresponding to the Bb protein, is estimated using the NIH ImageJ software (National Institutes of Health, USA).

To evaluate the effect of the purified IgGs on the decay of the C3bBb complex, some of the wells are also incubated, preferably for 32 minutes, in the presence of the buffer, so as to evaluate the spontaneous decay, or in the presence of FH, preferably 2.640 ng/mL, so as to evaluate the FH-mediated decay. After washing with the washing solution, we proceeded as indicated above to detach the remaining complexes and to the electrophoretic separation and subsequent detection of the bands. On the same gel, the baseline samples and the samples subjected to the subsequent incubation are run, with or without FH. The result obtained from the 60 kDa band intensity ratio after the decay and before the decay is indicative of C3NeF activity in the tested sample.

In a second embodiment, the method according to the present invention provides for the addition of said purified IgGs during the decay step. In said embodiment, said IgGs are not added during said step b) of complex formation.

Said second embodiment comprises the following steps:

a) immobilization of C3b in the wells of a plate;

b) addition in said wells of FB, FD, BSA, and magnesium ions;

c) in at least a first of said wells, complex formation and decay step comprising washing and incubation with IgGs purified from a biological sample from a subject in the absence and/or in the presence of FH and, in at least a second of said wells, complex formation and decay step comprising washing and incubation with IgGs purified from a control sample in the absence and/or in the presence of FH;

d) detachment of the formed complexes;

e) electrophoretic run in denaturing conditions of said complexes, with dissociation of the two components C3b and Bb, and transfer on a membrane;

f) detection of the protein of interest, Bb, to obtain a value referred to the samples obtained in the wells in which the decay step was carried out in the presence of IgGs isolated from the patient and a value referred to the samples obtained in the wells in which said decay step occurred in the presence of IgGs isolated from a control.

The method also comprises the measurement of the intensity ratio of said Bb band after the decay in the presence of the sample IgGs from the tested subject to the intensity of said Bb band after the decay occurred in the presence of control IgGs, with or without FH.

In a preferred embodiment, in said step c) at least two wells are prepared for the sample of the tested subject, where FH is added to one of the two wells and at least two wells for the control sample, where FH is added to one of the two wells.

The sample in which said ratio of the sample of the tested subject to the control sample is at least 1.6, or at least 1.7, preferably at least 1.9 in the absence of FH (spontaneous decay) and at least 2, or at least 2.1, preferably at least 2.3 is considered positive in the presence of FH (FH-mediated decay).

In a third embodiment, the method according to the present invention allows to evaluate the properdin combined effect. In this embodiment, during said step b) of the method according to the first embodiment, properdin is added to the well. The intensity ratio of the Bb bands before and after the decay is measured and the results are considered positive for values higher than 39%, or 40%, preferably 41%, or 42% in the absence of FH or above 8%, preferably 9%, or 10% in the presence of FH.

The scope of protection of the present invention is defined by the claims. The following examples have the sole purpose of showing some embodiments of the method according to the present invention and are in no way to be understood as limiting the scope of protection of the invention itself.

### EXAMPLE 1: ANALYSIS OF C3NEF ACTIVITY IN SAMPLES FROM HEALTHY OR PATHOLOGICAL SUBJECTS

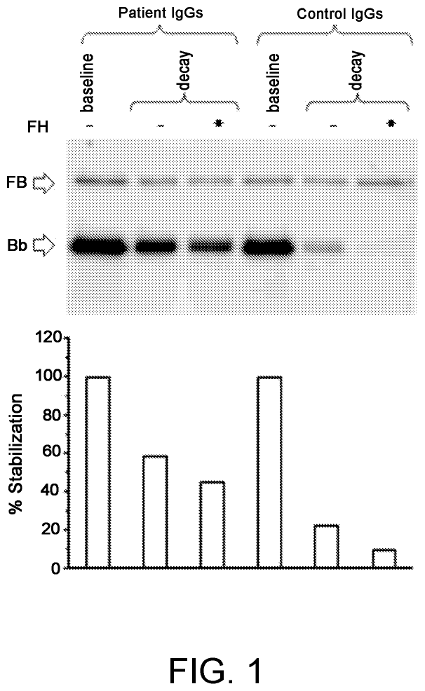

The C3 convertase complexes, C3bBb (Mg²⁺) were formed by incubating at 25° C. for 12 minutes the wells in which C3b was immobilized, with FB, FD and MgCl₂, in the presence of C3NeF-IgG purified from an MPGN patient (IgG patient) or from a healthy control (IgG control). The spontaneous or FH-mediated decay was monitored after further incubation for 32 minutes at 25° C. in the absence or in the presence of FH, respectively. The results of the WB analysis are shown in FIG. 1. The amount of formed C3 convertase was calculated as the ratio of the densitometry of the Bb band after the decay to the densitometry of the band corresponding to baseline*100.

Analyzing the mean±2 Dev Std of the results obtained by testing 35 samples from healthy donors, the samples in which a residual Bb band is observed after a decay higher than 36% with respect to the baseline, are considered positive where said decay occurred in the absence of FH, or higher than 11% with respect to the baseline, where said decay occurred in the presence of FH.

### EXAMPLE 2: ANALYSIS OF C3NEF ACTIVITY, WHERE IGGS ARE ADDED DURING THE DECAY PROCESS

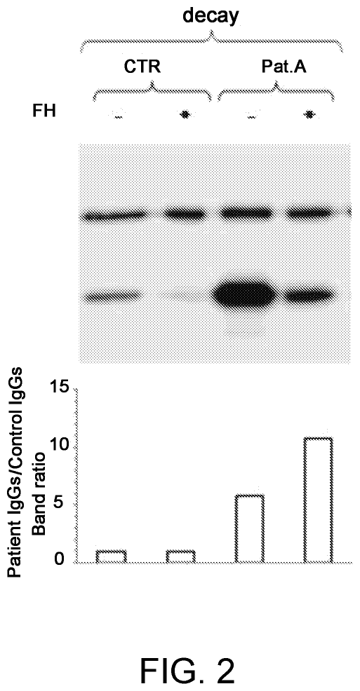

The C3 convertase complexes, C3bBb (Mg²⁺) were formed by incubating the wells in which C3b was immobilized with FB, FD and MgCl₂ at 25° C. for 12 minutes. Once formed, the complexes were incubated in the presence of C3NeF-IgG purified from an MPGN patient (pat A) or from a healthy control (CTR) and subjected to spontaneous or FH-mediated decay with further incubation for 32 minutes at 25° C. in the absence or in the presence of FH, respectively. The results of the WB analysis are shown in FIG. 2. The amount of formed C3 convertase was calculated as the ratio of the densitometry of the Bb band after the decay in the presence of the patient IgGs to the densitometry of the Bb band after the decay in the presence of control IgGs.

Analyzing the mean±2 Dev Std of the results obtained by testing 35 samples obtained from healthy donors, the samples in which the ratio of the sample of the tested subject to the sample of the control subject is higher than 1.9 in the absence of FH, and 2.3 in the presence of FH are considered positive.

### EXAMPLE 3: COMBINED EFFECT OF PROPERDIN

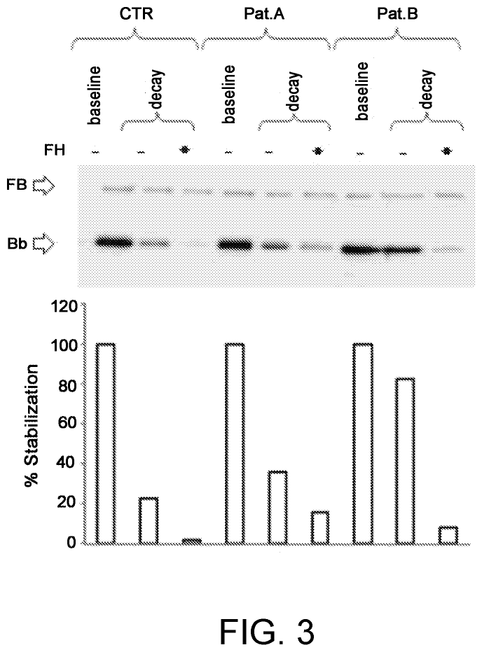

The C3 convertase complexes, C3bBb (Mg²⁺), were formed by incubating the wells in which C3b was immobilized with FB, FD, MgCl₂ and properdin in the presence of C3NeF-IgG purified from two MPGN patients (pat A, pat B) or from a healthy control at 25° C. for 12 minutes. (CTR). The spontaneous or FH-mediated decay was monitored by further incubation for 32 minutes at 25° C. in the absence or in the presence of FH, respectively. The results are shown in FIG. 3. The amount of formed C3 convertase was calculated as the ratio of the densitometry of the Bb band after the decay to the densitometry of the band corresponding to baseline×100.

Analyzing the average ±2 Dev Std of the results obtained by testing 35 samples from healthy donors, the samples in which a residual Bb band is observed after a decay higher than 41% with respect to the baseline, are considered positive, where the decay occurred in the absence of FH, or higher than 9% with respect to the baseline, where said decay occurred in the presence of FH.

1-11. (canceled)

12. A method for the diagnosis of C3NeF-mediated membranoproliferative glomerulonephritis (MPGN) in a subject comprising:
a) immobilization of C3b in at least two wells;
b) formation in said wells of the C3bBb complex wherein said formation of the complex is mediated by the addition of IgGs purified from a biological sample of the subject and/or control in the presence of Factor B (FB), Factor D (FD), magnesium ions;
c) detachment of the formed complexes;
d) electrophoretic run under denaturing conditions of said complexes and transfer on a membrane;
e) detection of the protein of interest, Bb;
wherein the ratio of the intensities measured for the Bb band in the samples collected by each of said wells is indicative of C3NeF activity in said subject.

13. A method according to claim 12, wherein in said step b) said wells further comprise Bovine Serum Albumin (BSA).

14. A method according to claim 12, comprising:
a) immobilization of C3b in at least two wells;
b) formation in said wells of the C3bBb complex, mediated by the addition of IgGs purified from a biological sample of the subject and/or control, in the presence of FB, FD, BSA, and magnesium ions;
c) detachment of the formed complexes;
d) electrophoretic run under denaturing conditions of said complexes and transfer on a membrane;
e) detection of the protein of interest, Bb, to obtain a value referred to as baseline;
wherein with respect to said step b) the addition of purified IgGs is followed, in one or more wells, including after a washing step, a further incubation in the presence or absence of FH, wherein the ratio of the intensity measured for the Bb band in the samples subjected to said further incubation to the baseline Bb band is indicative of C3NeF activity in said sample.

15. A method according to claim 14, wherein during said step b) properdin is further added.

16. A method according to claim 12, comprising:
a) immobilization of C3b in at least two wells;
b) addition in said at least two wells of FB, FD, BSA, and magnesium ions;
c) in at least a first of said wells, complex formation and decay step comprising washing and incubation with IgGs purified from said sample in the absence and/or in the presence of FH and, in at least a second of said wells, complex formation and decay step comprising washing and incubation with purified IgGs from a control in the absence and/or in the presence of FH;
d) detachment of the formed complexes;
e) electrophoretic run under denaturing conditions of said complexes and transfer on a membrane;
f) detection of the protein of interest, Bb, to obtain a Bb value referred to said at least a first well in which the decay step was carried out in the presence of IgGs isolated from said subject and a Bb value referred to said at least a second well in which said decay step occurred in the presence of IgGs isolated from a control,
wherein the ratio of the intensity of said Bb band referred to said at least a first well in which the decay step was carried out in the presence of IgGs isolated from said subject to the intensity of said Bb band referred to at least a second well in which said decay step occurred in the presence of IgGs isolated from a control, with or without FH, is indicative of C3NeF activity in said subject.

17. A method according to claim 12, wherein said sample is a serum sample.

18. A method according to claim 12, wherein said detection is carried out using an anti-human FB antibody.

19. A method according to claim 14, wherein values of the Bb band intensity after said further incubation equal to at least 34%, or at least 35%, or at least 37% with respect to the values measured in the baseline of the same sample are indicative of positivity of said subject for C3NeF-mediated MPGN.

20. A method according to claim 14, wherein values of the Bb band intensity after further incubation in the presence of FH equal to at least 9%, or at least 10%, or at least of 12% with respect to the values measured in the baseline of the same sample are indicative of positivity of said subject for C3NeF-mediated MPGN.

21. A method according to claim 15, wherein values of the intensity ratio of Bb bands before and after the decay higher than 39%, or 40%, or 41%, or 42% in the absence of FH or above 8%, or above 9%, or above 10% in the presence of FH are indicative of positivity of said subject for C3NeF-mediated MPGN.

22. A method according to claim 16, wherein values of said ratio equal to at least 1.6 or at least 1.7, or at least 1.9 in the absence of FH and at least 2, or at least 2.1, or at least 2.3 in the presence of FH are indicative of C3NeF-mediated MPGN.
